(19) United States
(12) Patent Application Publication
(10) Pub. No.: US 2018/0369003 A1
(43) Pub. Date: Dec. 27, 2018

(54) RADIALLY SELF-EXPANDABLE ROLLED UP TUBULAR STENT

(71) Applicants: COUNCIL OF SCIENTIFIC & INDUSTRIAL RESEARCH, New Delhi (IN); EMBRYYO TECHNOLOGIES PVT.LTD., Maharashtra (IN)

(72) Inventors: Guruswamy KUMARASWAMY, Maharashtra (IN); Sayam Sen Gupta, Maharashtra (IN); Bhagavatula Lakshmi Vara PRASAD, Maharashtra (IN); Nishant KUMAR, Maharashtra (IN); Prateek JAIN, Maharashtra (IN)

(21) Appl. No.: 15/775,455

(22) PCT Filed: Nov. 10, 2016
(86) PCT No.: PCT/IN2016/050394

§ 371 Date: May 11, 2018

(30) Foreign Application Priority Data
Nov. 11, 2015 (IN) 2463/DEL/2015

(51) Int. Cl. A61F 2/92; A61F 2/962

(52) U.S. Cl. CPC A61F 2/92; A61F 2/962; A61F 2210/0004; A61F 2220/0033; A61F 2230/0019; A61F 2230/0023; A61F 2230/0069

(57) ABSTRACT

The present invention discloses a rolled up radially self-expanding stent comprising at least one transverse expandable member of non-shape memory alloy sheet or wire loops laminated in a polymer and rolled up in a configuration that stores elastic energy and retains flexibility, arranged on a longitudinal axis used to improve the lumen patency of non-vascular body lumens. The radial expansion of the stent is caused by unrolling of the rolled up transverse expandable member during deployment in the body lumen.

The following specification particularly describes the invention and the manner in which it is to be performed:

### FIELD OF THE INVENTION

The present invention relates to a rolled up radially self-expanding stent comprising at least one transverse expandable member of non-shape memory alloy sheet or wire loops laminated in a polymer and rolled up in a configuration that stores elastic energy and retains flexibility, arranged on a longitudinal axis used to improve the lumen patency of non-vascular body lumens. The radial expansion of the stent is caused by unrolling of the rolled up transverse expandable member during deployment in the body lumen.

### BACKGROUND AND PRIOR ART

The implantation of tubular prostheses to replace damaged or diseased vascular vessels or other luminal passageways within the human body is known in the art. In applications, such as delivery of tubular medical devices into remote portions of the human vasculature, the tubular structure should be capable of passing through the narrow vasculature. Therefore, the wall thickness and the overall profile of the tubular device have become increasingly important for overall procedure and comfort of the patient.

Stents are tubes used to treat atherosclerotic stenosis or other type of blockages in body lumen like blood vessels, oesophagal duct, biliary duct by expanding the lumen that has narrowed due to disease. Stents known in the art can be balloon expandable, self-expanding or are hybrid expandable.

Self-expanding stents can be inserted in body lumen in a “crimped” state and subsequently expanded radially to increase their diameter to expand the passage for flow of bodily fluids. Several designs for self-expanding stents exist in the patent literature. These include rolled up cylinders, spirally wound springs, web structures, helically coiled ribbons, as coiled sheets etc. The majority of self-expanding metallic stents are used to alleviate symptoms caused by cancers of the gastrointestinal tract that obstruct the interior of the tube-like (or luminal) structures of the bowel like esophagus, duodenum, common bile duct and colon. Self-expanding stents are not used for all applications. For example, cardiovascular stents are not self-expanding stents.

Stents are manufactured either from a single material such as metal or polymer by laser or chemical machining. The properties of the stent are dependent on the material used. For instance, if the stent is prepared using only metal the stents have relatively high strength, stiffness but are less elastic in nature as compared to stents made of polymer which have more axial and radial flexibility.

There are prior arts which disclose the use of combination of composite materials for self-expanding stents. The materials include expensive shape memory alloys such as nitinol, other metal alloys, platinum, gold, textiles, polymers such as polytetrafluoroethylene, expanded polytetrafluoroethylene, plastics however, are either not axially flexible (plastic stents) or change axial size on coiling (spirally wound springs) or will form kinks on bending. Therefore, these designs cannot be used for applications where lumen patency is required.

There are reports which suggest that the fixed diameter plastic biliary stents suffer from major drawback that they get clogged rapidly compared to expandable memory alloy based stents. The maximum diameter of such plastic stents is limited by the stenting tool. As this is limited to about 3 mm diameter, plastic stents experience rapid blockage on implantation in the body, relative to self-expanding stents.

U.S. Pat. No. 5,833,699 discloses spirally wound metal coil with pattern cuts to allow for further expansion and the adjacent spiral wounds connected to each other with struts to preserve the tubular structure on expansion of the spirally wound coils. The expansive forces of such a stent are typically reduced along the length of the stent; resistance to radial compressive forces are low; the stents made are balloon expanding and not self-expanding and therefore internal stresses in the stent built up in the struts connecting the adjacent spiral layers as the stent expands to the unrestrained state.

The use of balloon catheters is not desirous since the balloons can burst due to pressure during deployment which can in turn lead to rupture of the lumen. For longer stents/grafts, expanding with balloon might not be practical as the balloon would have to be repositioned along the entire length of the stent which may reduce lumen patency.

U.S. Pat. No. 6,090,136 relates to Stainless steel coils deployed one by one on a deployment catheter within a flexible tubular support. In this case the tubular support can expand only piecewise, at the location where the coiled metal sheet is deployed.

U.S. Pat. No. 5,723,003 discloses polymer (PTFE) tube that is sandwiched between outer rolled metal structure and a separate inner rolled metal structure. Expansion of the polymer stent is achieved by the inner metal structure while the outer metal structure anchor to the walls of the body lumen. Two independent rolled structures (on the inside and outside) need to be used to effect expansion to fit the body lumen. In this case, the force exerted by the outer metal structure directly on the body lumen could result in edema.

US2004/0254630 describes self-expanding stent with a combination of coiled sheet configuration and stretchable zig-zag patterns. Said stent is made of shape memory alloy and a manufacturing step which requires imparting of shape memory to the said sheet by heat treatment or annealing. The heat treatment or annealing involved in such processes can lead to brittleness around the joint or reduction of the resilient nature of such a wire or segment.

U.S. Pat. No. 6,001,123 relates to self-expanding stent based on metal (stainless steel) and polymer and folded so that it can expand from a folded to unfolded state. The self-expansion is based on folding of an initially tubular structure. This approach has limitations since it can result in kinking/plastic deformation of the metal at the points with smallest radius of curvature in the folded state. Folding will necessarily involve localized segments that have a small radius of curvature.

Therefore, there is an acute need in the art for cost effective self-expandable tubular stents which stores the elastic energy and flexibility in a rolled up configuration and lends anti-migration property when the stent radially self-expands with no localized deformation along its longitudinal axis on deployment in the body lumen.

### OBJECTIVE OF THE INVENTION

The primary object of the invention is to provide stent which stores the elastic energy and flexibility in a rolled up configuration which radially self-expands with no localized deformation along its longitudinal axis on deployment in the body lumen.

The other object is to provide stent that does not use expensive shape memory alloys and does not require complex manufacturing strategies.

### SUMMARY OF THE INVENTION

Accordingly, the improvements in the present invention include providing axially flexible, radially expandable rolled up tubular stent, to improve the lumen patency of non-vascular body lumens, comprising at least one expandable member consisting of non-shape memory alloy sheet or wire loops laminated in a polymer and rolled up in a configuration that stores elastic energy and flexibility, with a plurality of such members arranged on a longitudinal axis of said stent. The radial expansion comes from a rolled up design that expands radially as the tube unrolls.

In an aspect, the plurality of said transverse expandable members are minimally connected with each other via the polymer layer alone or by virtue of being a continuous sheet or a continuous pattern of wire frames enclosed in a polymer layer or by a flexible wire on which each of the transverse expandable members are arranged sequentially with the flexible wire passing freely through loops made on the said transverse expandable members.

In another aspect, each expandable member is provided with a locking feature.

In an aspect, the transverse expandable members have varying length, width and/or thickness producing varying radial forces on the lumen walls and/or a varying outer primary diameter along the longitudinal axis of the stent.

In another aspect, the present invention provides a method of rolling non-shape memory alloy sheet or wire loops laminated in a polymer to obtain rolled up tubular stent which is axially flexible and radially expandable wherein the radial expansion comes from a rolled up tube design that expands radially as the tube unrolls.

### BRIEF DESCRIPTION OF THE DRAWINGS

FIG. 1 depicts un-connected parallel rectangular metal strips

FIG. 2 depicts one-edge connected parallel rectangular metal strips

FIG. 3 depicts both edges connected parallel rectangular metal strips

FIG. 4 depict (a) Metal strips enclosed in polymer; (b) Metal sheet enclosed in a polymer with several transverse expandable members free to be coiled individually; (c) Metal wire loop enclosed in the polymer.

FIG. 5 depict (a) Multiple transverse expandable members laminated in the polymer connected with each other by the virtue of being a continuous sheet; (b) wire loops instead of metal sheets; (c) the multiple transverse expandable members assembled on a flexible wire to make them interconnected.

FIG. 6 depict self-locking feature.

FIG. 7 depict the migration prevention feature of the stent wherein coiled metal polymer sheets is extended to the top and bottom ends of the stent such that they lend anti-migration capability to the stent when deployed in the body lumens. In figure (a), the coiled sheets on the extremities uncoil to a diameter larger than the expanded state diameter of the stent in the transverse direction while in the figure (b), the metal polymer sheets are placed in a way such that that they uncoil to a larger diameter at the top and bottom ends in the longitudinal direction.

FIG. 8 depict radial expansion (a) without locking system; (b) with locking system.

FIGS. 9 (a)-(d) depict the various embodiments of automated rolling method of the stent.

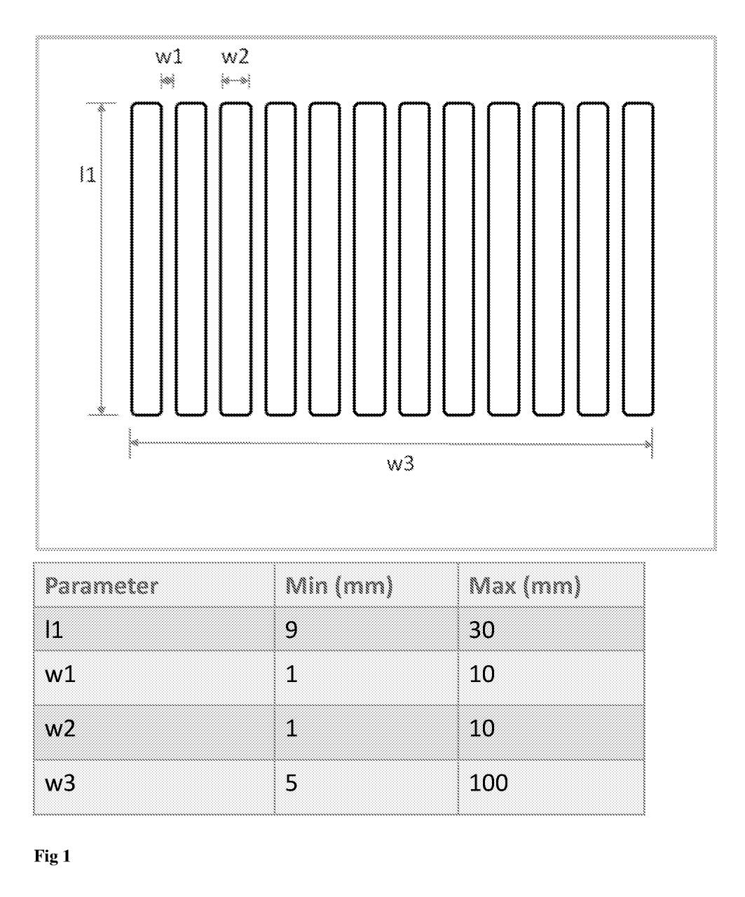

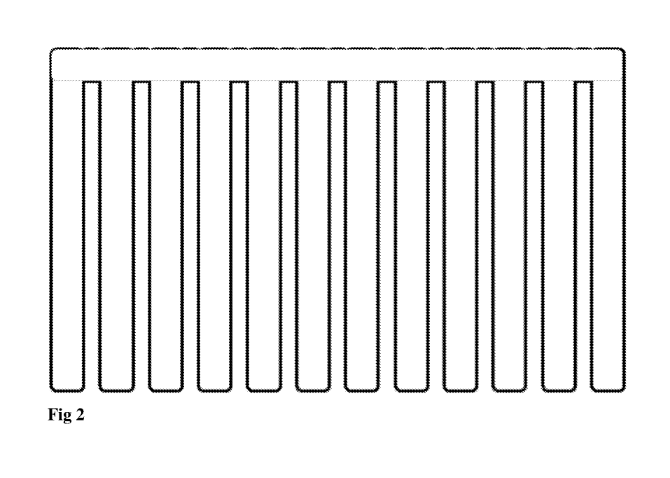

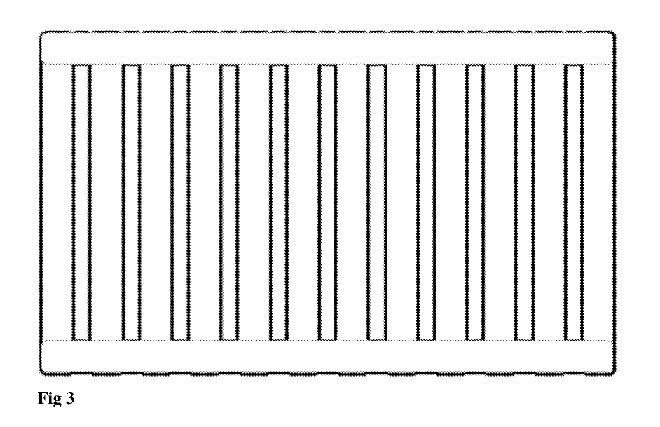

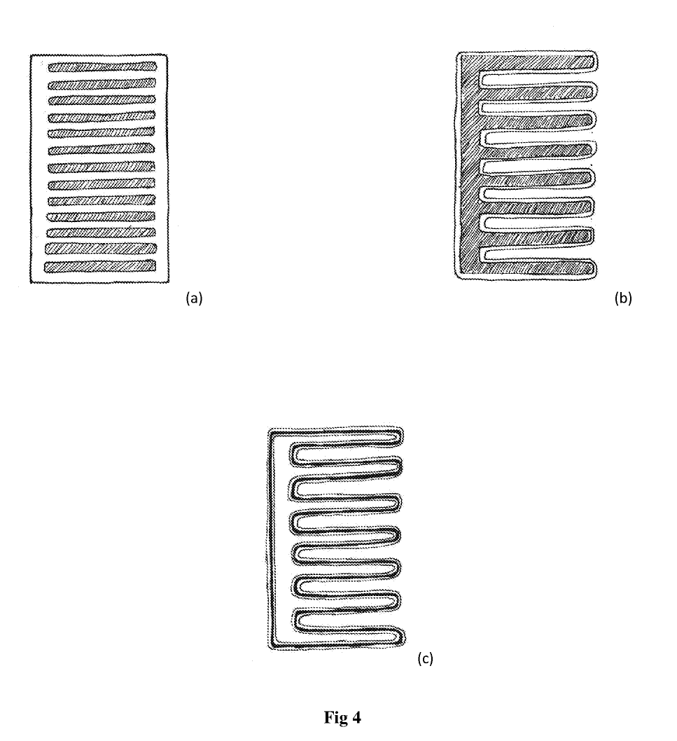

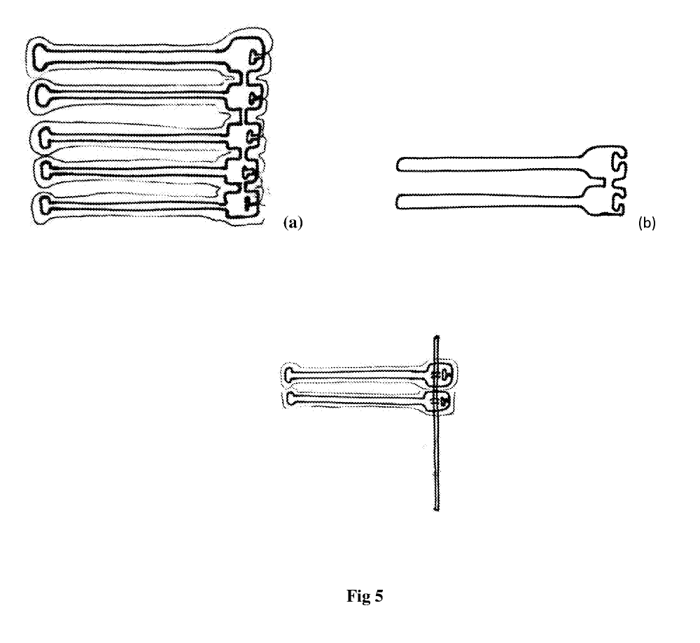

### DETAILED DESCRIPTION OF THE INVENTION

The invention will now be described in detail in connection with certain preferred and optional embodiments, so that various aspects thereof may be more fully understood and appreciated.

Tortuous lumen leads to over-deformation/stresses in the body of the stent, which happens primarily because of the interconnections between the transverse expanding elements and the longitudinal connecting elements. Further, foreshortening of the stents creates inconvenience during deployment since the inaccurate placement can lead to the need for re-positioning. For esophageal application the problem is more pronounced as the endo-luminal prosthesis must also reciprocate the forces due to peristalsis for maximum patient comfort. Esophageal stenting near the trachea compromises respiration, near gastrointestinal (GI) junction, the stent is more prone to migration and to improve lumen patency stents which are stable, shape conforming throughout the body lumen on expansion from the constricted diameter to the expandable diameter are provided in the present invention.

In an embodiment, the present invention relates to a rolled up radially self-expanding tubular stent comprising at least one transverse expandable member consisting of non-shape memory alloy sheet or wire loops laminated in a flexible bio-absorbable polymer and rolled up in a configuration that stores elastic energy and flexibility, with a plurality of such members arranged on a longitudinal axis of said stent, wherein the radial self-expansion is caused from a rolled up tubular stent design that expands radially as the tube unrolls.

The stent is constricted to a smaller diameter by rolling such that there is a cylindrically repeating overlap between the inner and the outer edges of the transverse expandable member. The stent is retained in said constricted diameter using an outer sheath selected from a polymer, which is removed during stent deployment in the body lumen. On deployment, the removal of the outer sheath allows the transverse expandable member to radially self-expand to a diameter larger than the initial diameter, remaining constrained within a non-removable/coated inner sheath.

The non-shape memory alloy sheet or wire loops is selected from any biocompatible metal consisting of stainless steel, aluminium, titanium alloys, tantalum alloys, nickel alloys, cobalt alloys or chromium alloys; preferably the metal used is stainless steel.

The ratio of diameters of said stent in the fully expanded state to the initial coiled up state is in the range of 2 to 6.

In an embodiment, the sequential arrangement of said transverse expandable members on said longitudinal axis precludes foreshortening when the stent expands from an initial constricted diameter to the primary outer diameter. The stent remains placed in the body lumen even on expansion and avoids the need for re-positioning.

The flexible bio-compatible polymer for lamination Polyethylene terephthalate as well as for use as outer sheath is selected from the group consisting of polyethylene (PET), poly tetra fluoro ethylene (PTFE), silicone, polyurethane, poly(L) lactic acid (PLLA), PCL (polycaprolactone), PLGA (poly-L-glutamic acid), PHBV (polyhydroxybutyrate-co-valerate) polyisoprene or polybutadiene, polysaccharides including cellulose, chitin, dextran, starch, hydroxyethyl starch, polygluconate, hyaluronic acid; polyamides including polyamino acids, polyester-amides, polyglutamic acid, poly-lysine, gelatin, fibrin, fibrinogen, casein, collagen, polyesters including poly-alpha hydroxy and poly-beta hydroxy polyesters, polycaprolactone, polyglycolic acid, polyether-esters and other such bioabsorbable polymers. Preferably, the metal sheet is coated with silicone and the outer sheath is polyethylene.

The flexible polymer has a thickness varying between 20 to 200 microns. The thickness of the non-shape memory alloy sheet used in the said transverse expandable member is 10 to 5000 microns whereas the polymer covered metal strip or sheet possesses thickness varying from 25 to 8000 microns. The diameter of the metal wire used in the said transverse expandable member is in the range of 10 to 1000 microns.

In another embodiment, the said transverse expanding members comprising non-shape memory alloy sheets and enclosed with polymer to achieve lumen patency and maximum patient comfort. The length, width and/or thickness of each of the transverse expandable members varies along the longitudinal axis contributes to varying radial forces on the lumen walls and/or a varying outer primary diameter along the longitudinal axis of the stent.

The plurality of said transverse expandable members is minimally connected with each other either as the polymer layer alone or by virtue of being a continuous sheet or a continuous pattern of wire frames enclosed in a polymer layer or a flexible wire on which each of the transverse expandable members are arranged sequentially with the flexible wire passing freely through loops made on said transverse expandable members. The minimal interconnections lead to lesser stress build up in the body of the stent.

The rolled up self-expandable tubular stent has a first end and a second end having at least one transverse expandable member or a plurality of them along its longitudinal axis which expands radially as the tube unrolls defines lumen there through. The expandable member has an outer surface which is the abluminal surface of the stents composite and the inner surface is the luminal surface of the stent composite.

The flexible wire on which the at least one transverse expandable member or a plurality of them are sequentially arranged comprising non-shape memory alloy can be pre-formed in different shapes.

The self-expandable member of the tubular stent is defined by individual struts/elements of varying thickness in the transverse direction so that each such strut/element can produce different radial forces along the length of the stenosis consisting of non-shape memory alloy sheet or wire loops laminated in a polymer and rolled up in a configuration along its width to constrain the strut in a pre-determined smaller radius that stores elastic energy and flexibility. Alternately, the patterns or cuts may be made in the polymer region alone of the strut.

The patterns in the metal sheet or mesh may comprise an un-connected parallel rectangular metal strips, one-edge connected parallel rectangular metal strips, both edges connected parallel rectangular metal strips, zig-zag connected parallel rectangular metal strips, combination of connected and un-connected connected parallel rectangular metal strips, parallel rectangular metal strips connected with round edges, angular metal strips connected with round edges, un-connected parallel rectangular metal strips placed at an angle with respect to the axis of scroll rotation, Metal sheet may have diamond cuts, rectangular cuts, circular cuts, composite pattern cuts, unconnected triangular mesh cuts, connected triangular mesh cuts, connected triangular mesh cuts with unconnected boundaries, with un-connected modules of triangular mesh cuts flipped alternately. The pattern cuts into the metal sheet provide axial flexibility along with the possibility of lumen patency.

In an aspect, the strut can be wire loop as shown in having a multitude of such parallel layers. The strut in the form of wire loop has the advantage that it minimizes torsional effects in the stent due to tortuous lumen geometries.

The stent may have a length varying from 2-20 cm. The dimensions of the struts that define the sheet to fabricate tubular stent structure are varied depending on its application, the area to be treated and the needs of the patient. Accordingly, the rectangular metal strips have a length in the range of 9 mm to 75 mm and the width can vary from 1 mm to 100 mm; the metal strips connected with round edges have an angle of about 45°; the unconnected parallel rectangular metal strips placed at an angle with respect to the axis of scroll rotation has 02 in the range of 45-90°; the metal sheets with diamond cuts have a width ranging from 1 mm-10 mm; the metal sheets with circular cuts have a width ranging from 1 mm-25 mm; the metal sheets with unconnected triangular mesh cuts have a width in the range of 1 mm-10 mm and the angle with respect to the axis of scroll rotation has 03 in the range of 10-180°; the width of metal sheet with connected triangular mesh cuts is in the range of 5-95 mm. The angle of the pattern, the degree of overlap, the circumference of the sheet all contribute to the axial flexibility of the stent along its longitudinal axis.

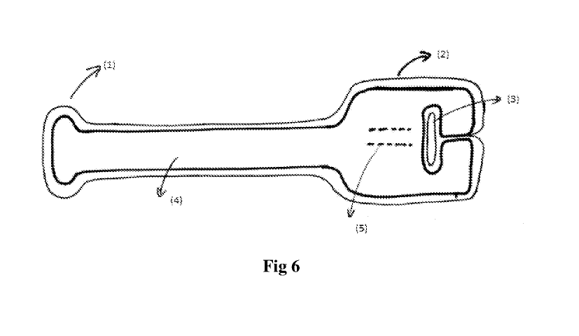

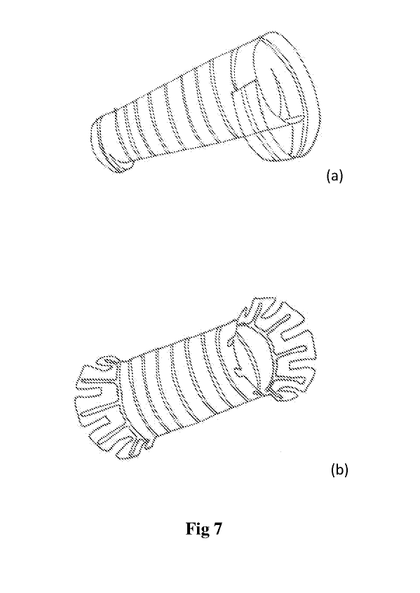

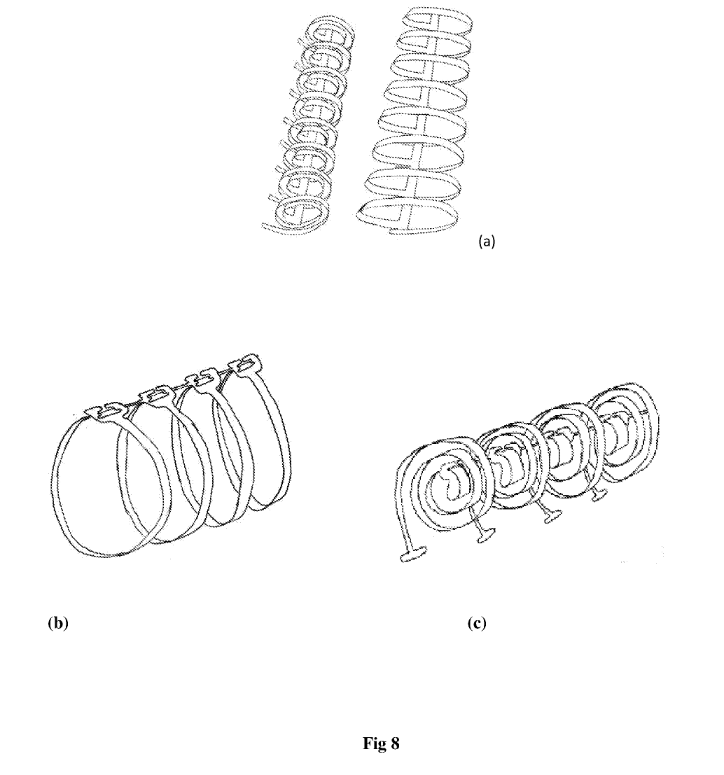

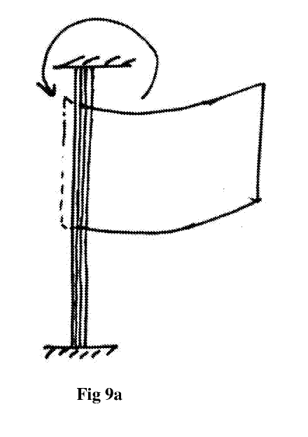

In an embodiment, each expandable member has a locking feature as shown in FIG. 6. Accordingly, the transverse expandable member comprising non-shape memory alloy sheet or wire loop laminated in a polymer has a broadened tip on one end and a T-shape slot cut on the other end. There is also a horizontal cut made on the sheet through which a flexible wire can pass in the longitudinal direction as per one of the embodiments of interconnection between the transverse expanding members. The two ends of the expandable member are bent at an angle in the range of 30 to 45° such that on uncoiling the two ends overlap to form a complete circle. In the coiled state, the broadened tip of the expandable member is inserted through the T-slot only once and then it is further coiled up over itself multiple times to achieve the elastic energy storing coiled configuration.

In an another embodiment, where there is no lock in the strut, the two edges of the strut are individually coiled up so that when they uncoil the rounded edges push onto each other keeping the uncoiled strut circularly closed.

In an embodiment, the top and bottom ends of a stent having similar elastic energy storing coiled up expandable structures either in the transverse or longitudinal directions, constrained within the outer sheath opening to a secondary outer diameter larger than the primary outer diameter of the overall stent, on the removal of the outer sheath, ensures prevention of migration of the stent when deployed in the body lumen.

The arrangement of the struts in the expandable member provides tubular, axially flexible stent assembly where each individual strut or all such struts as a whole can self-expand to a larger outer diameter from an initial smaller diameter through a mechanism involving un-coiling of a coiled/scrolled tubular sheet structure.

In an aspect, the stent of the present invention in the fully expanded state when bent in a direction orthogonal to the longitudinal axis through an angle of up to 60° reassumes the original longitudinal orientation on removing the axial bending forces. Further, the axial bending force does not produce a kink in the body of the stent and retains high flexibility in the expanded state.

In an embodiment, the stent is open ended and uncoils to a larger diameter at the top and bottom ends in the longitudinal direction with no foreshortening.

In a preferred embodiment, the present invention provides a rolled up radially self-expanding tubular stent, to improve the lumen patency of non-vascular body lumens, that stores elastic energy and retains flexibility in a rolled up configuration comprising;
at least one transverse expandable member consisting of stainless sheet or wire loops laminated in silicone polymer, with a plurality of such members arranged on a longitudinal axis of said stent;
an outer sheath of polyethylene polymer that retains the stent in a constricted diameter prior to deployment;
multiple strain-relieving patterns cut along the length of plurality transverse expandable members of varying length, width and thickness wherein the plurality transverse expandable members are minimally connected with each other either as the silicone layer alone or by virtue of being a continuous sheet or a continuous pattern of wire frames enclosed in silicone layer or a flexible wire on which each of the transverse expandable members are arranged sequentially with the flexible wire passing freely through loops made on said transverse expandable members;
a locking feature wherein the broadened tip of the expandable member is inserted through the T-slot only once and is further coiled up over itself multiple times to achieve the elastic energy storing coiled configuration;
   wherein the stent is open ended and uncoils to a larger diameter at the top and bottom ends in the longitudinal direction with no foreshortening.

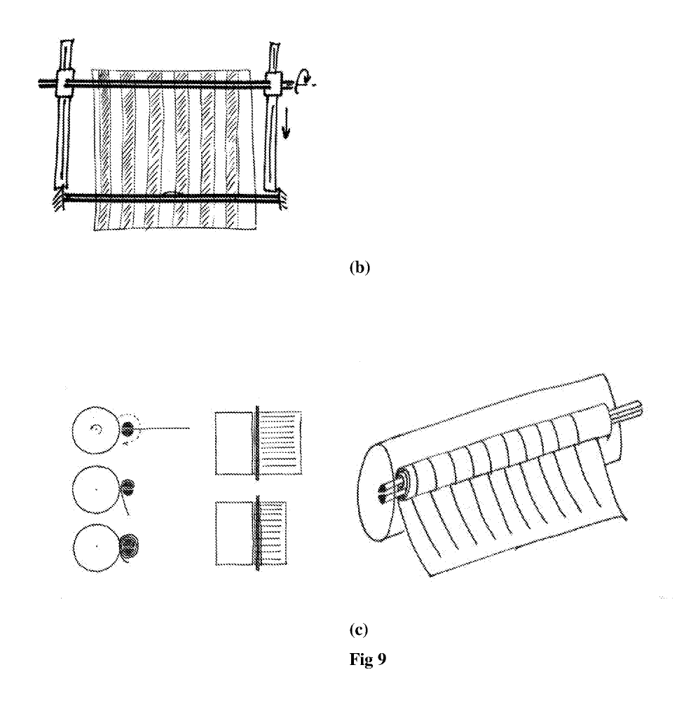

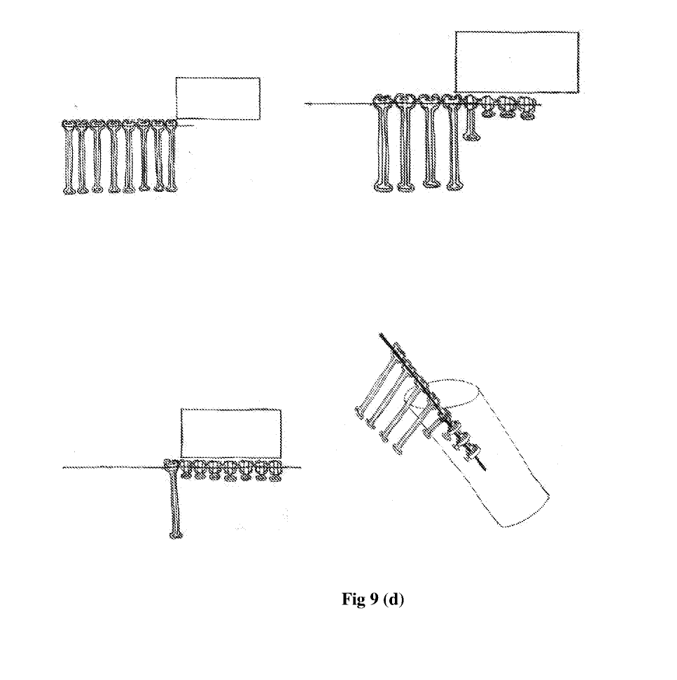

In another embodiment, the present invention relates to a method of rolling non-shape memory alloy sheet or wire loop laminated in a polymer along the longitudinal axis of the self-expanding stent tubular structure using taut wires which renders it axially flexible and radially expandable wherein the radial expansion comes from a rolled up tubular design that expands radially as the tube unrolls. As an illustration (FIG. 9a), the sheet metal of stainless steel (SS316) of about 30 microns is taken and is cut in a pattern using wire EDM process with wire thickness of about 200 microns. The patterned cut sheet is coated with silicone material (MED 4860) which comprises pouring the silicone material on the metal sheet using caulking gun. The whole assembly with silicone material and metal is placed between polyethylene (PET) sheets and then placed in the hot press. The pressure of about 2000 psi is applied and the temperature is raised up to 125° for about 90 seconds. The assembly is cooled naturally for about 180 seconds, the PET sheets are removed, the edges/flash material is further removed to obtain metal sheet coated with polymer.

Further, the metal sheet coated with polymer of the dimension approx. 110 mm×70 mm (L×W) are taken, two metal wires of diameter approx. 1.5 mm each are positioned in a taut condition on a fixating jig and a polymer outer sheath approx. of diameter 6 mm and 0.1 mm sheet thickness are inserted in the taut wires and the metal sheet coated with polymer are inserted between taut wires up to a depth of about 1 mm. The sheet is rotated about the axis of taut wires in the form of a coil and the rolling of the sheet is stopped when the complete assembly reaches diameter of approx. 5.5 mm. The assembly is inserted into a approx. 6 mm outer sheath, the taut wires are released and the rolled sheet is taken out axially.

In another embodiment, the sheets may be rolled automatically (FIG. 9b), wherein one end of the sheet is fixed between two static rods and the other end of the sheet is clamped between another pair of rods. The said pair of rods rotates on its axis winding the sheet and the winding assembly is further assembled on a pair of rails. As the sheet gets wound, the free end of parallel rods move closer to the other fixed end. After the winding process, the sheets are released from the rods mechanically or by other means.

Alternately, the automated method of rolling (FIGS. 9(c) and (d)) comprises clamping the metal sheet with the metal rod such that all elements are connected to the metal rod, the whole assembly is placed near the roller and fixed such that the metal rod can rotate about its axis and can move linearly along the axis, the assembly is then moved such that one of the element comes in contact with the cylinder, the assembly is rotated in clockwise direction about the axis of rod and cylinder rotated in anti-clockwise direction about its own axis wherein the individual element is spirally rolled to the desired outer diameter. The assembly is further moved linearly along the axis of rod, such that second element comes in contact with the cylinder and the assembly is rotated and the elements are scrolled to desired outer diameter. The process is continued till all the elements are scrolled to desired outer diameter to obtain the coiled up tubular stent.

In an embodiment, the rolled up radially self-expanding tubular stent comprising at least one transverse expandable member consisting of non-shape memory alloy sheet or wire loops laminated in the polymer and rolled up in the outer polymer sheath are preferably used in the esophageal, gastrointestinal and biliary regions, however, their use in other locations are contemplated according to the need of the patient.

In another embodiment, the method of deploying the rolled up radially self-expanding tubular stent comprises, introducing the rolled up stent in to the desired body lumen of the patient through a deployment catheter, wherein removal of outer polymer sheath allow the transverse member to radially self-expand to a diameter larger than the initial diameter, remaining constrained within a non-removable inner sheath on uncoiling/scrolling of the tubular coiled member, as the tube unrolls or uncoils.

The said transverse expandable member and a plurality of them in the fully expanded state resist external compressive forces and provide stability to the stent.

The present invention provides a one-time deployment of the entire stent as compared to multiple coiled elements being deployed one after the other in the art. Thus, the ease of use of the rolled up self-expanding stent of the present invention is much higher and provides lumen patency and comfort to the patient. The stents do not undergo any folding in the contracted state, there is no localized large deformation along the stent that might lead to plastic yield.

In an embodiment, the rolled up tubular stent of the present invention comprising at least one transverse expandable member consisting of non-shape memory alloy sheet or wire loops laminated in the polymer and rolled up in the outer polymer sheath which stores the elastic energy through an optimized rolling process helps in radial self-expansion through a mechanism involving un-coiling of a coiled/scrolled tubular sheet structure. The sandwiched configuration wherein the metal sheet or loop is laminated with a polymer requires only one kind of transverse rolled up structure (simpler construction to achieve the same result) and does not allow direct contact of the metal element with the body lumen. The stents of the present invention retain the simplicity of the rolled up tube design combined with the use of inexpensive, easy to fabricate composite material. The stents are relatively inexpensive to prepare (since they neither use expensive nitinol alloy nor complicated fabrication strategies for preparation).

It can be seen form the foregoing that while the various preferred and alternate embodiments illustrate the invention, those skilled in the art may recognize the other equivalents to the specific embodiment described herein which equivalents are intended to be encompassed by the appended claims.

1. A rolled up radially self-expanding tubular stent, to improve lumen patency of non-vascular body lumens, comprising:
at least one transverse expandable member consisting of non-shape memory alloy sheet or wire loops laminated in a flexible polymer and rolled up in a configuration that stores elastic energy and retains flexibility;
wherein the at least one transverse expandable member is arranged on a longitudinal axis of said stent; and
wherein the radial self-expansion is caused from a rolled up tubular stent design having a tube that expands radially as the tube unrolls.

2. The rolled up stent according to claim 1, wherein said stent is retained in a constricted diameter using an outer sheath selected from a flexible polymer prior to deployment in the body lumen.

3. The rolled up stent according to claim 1, wherein a plurality of transverse expandable member are sequentially arranged on said longitudinal axis to preclude foreshortening when the stent expands from an initial constricted diameter to a primary outer diameter.

4. The rolled up stent according to claim 1, wherein radial self-expansion upon un-coiling of a coiled/scrolled tubular sheet structure is caused due to elastic energy stored in the rolled up configuration of the at least one expandable member either in the transverse or longitudinal directions, as constrained within an outer sheath.

5. The rolled up stent according to claim 1, wherein said at least one transverse expanding members comprises multiple strain-relieving patterns cut along variable length, width and thickness along the longitudinal axis contributing to varying radial forces on the lumen and/or a varying outer primary diameter along the longitudinal axis of the stent.

6. The rolled up stent according to claim 4, wherein the plurality of said transverse members are minimally connected with each other either as the flexible polymer alone or by virtue of being a continuous sheet or a continuous pattern of wire frames enclosed in the flexible polymer or a flexible wire on which each of the transverse expandable members are arranged sequentially with the flexible wire passing freely through loops made on said transverse expandable members.

7. The rolled up stent according to claim 1, wherein said tranverse expandable member has a locking feature wherein a broadened tip of the expandable member is inserted through a T-slot only once and is further coiled up over itself multiple times to achieve an elastic, energy storing, coiled configuration.

8. The rolled up stent according to claim 1, wherein the stent is open ended and uncoils to a larger diameter at top and bottom ends in the longitudinal direction.

9. A rolled up radially self-expanding tubular stent that stores elastic energy and retains flexibility in a rolled up configuration, to improve the lumen patency of non-vascular body lumens, comprising;
at least one transverse expandable member consisting of stainless sheet or wire loops laminated in silicone polymer, with a plurality of such members arranged on a longitudinal axis of said stent;
an outer sheath of polyethylene polymer that retains the stent in a constricted diameter prior to deployment;
multiple strain-relieving patterns cut along the length of plurality transverse expandable members of varying length, width and thickness wherein the plurality transverse expandable members are minimally connected with each other either as the silicone layer alone or by virtue of being a continuous sheet or a continuous pattern of wire frames enclosed in silicone layer or a flexible wire on which each of the transverse expandable members are arranged sequentially with the flexible wire passing freely through loops made on said transverse expandable members;
a locking feature wherein the broadened tip of the expandable member is inserted through the T-slot only once and is further coiled up over itself multiple times to achieve the elastic energy storing coiled configuration; and
wherein the stent is open ended and uncoils to a larger diameter at the top and bottom ends in the longitudinal direction with no foreshortening.

10. A method of deploying the rolled up radially self-expanding tubular stent according to claim 1 comprising:
introducing the rolled up stent in to the desired body lumen of the patient through a deployment catheter
wherein removal of outer polymer sheath allow the transverse member to radially self-expand to a diameter larger than the initial diameter, remaining constrained within a non-removable inner sheath on uncoiling/scrolling of the tubular coiled member, as the tube unrolls or uncoils.
